(19) United States
(12) Patent Application Publication
(10) Pub. No.: US 2014/0057993 A1
(43) Pub. Date: Feb. 27, 2014

(54) Prophylaxis and Treatment of Enteropathogenic Bacterial Infection

(71) Applicant: NORTHSHORE UNIVERSITY HEALTHSYSTEM, Evanston, IL (US)

(72) Inventors: Suzana Savkovic, Oak Park, IL (US); Hemant K. Roy, Highland Park, IL (US)

(73) Assignee: NORTHSHORE UNIVERSITY HEALTHSYSTEM, Evanston, IL (US)

(21) Appl. No.: 14/114,206

(22) PCT Filed: Apr. 27, 2012
(86) PCT No.: PCT/US12/35493

§ 371 Date: Oct. 26, 2013

Related U.S. Application Data
(60) Provisional application No. 61/479,452, filed on Apr. 27, 2011.

(51) Int. Cl. A61K 31/77; A61K 45/06

(52) U.S. Cl. CPC A61K 31/77; A61K 45/06
USPC 514/723; 568/623

(57) ABSTRACT

In accordance with the present invention, methods are provided for the prevention and/or treatment of enteropathogenic bacterial infection in the gastrointestinal tract of a subject, and the diarrhea associated with the infection, by administration to the subject of a low molecular weight polyethylene glycol, as well concurrent administration with other antibiotic and antidiarrheal agents. Methods for reduction or suppression of inflammation, and inhibition of β1-integrin expression in the gastrointestinal mucosa are also provided. Also described is a kit suitable for use with the methods disclosed.

### BACKGROUND OF THE INVENTION

Enteropathogenic bacterial diarrhea is a major world health problem and one of the leading killers of children in the developing world. It is estimated that 1.9 million children worldwide die every year due to complications from enteropathogenic bacterial infection.

Enteropathogenic infections are prominent in developing nations and their impact has dramatically increased in the past decades in industrialized countries due to increase food processing and ease of worldwide travel. Virulent strains of Escherichia coli (E. coli) such as enteropathogenic E. coli (EPEC), enterotoxigenic E. coli (ETEC), enterinvasive E. coli (EIEC) and enterohemorrhagic E. coli (EHEC) are responsible for a significant proportion of bacterial enteric infections. While infections with EPEC cause infantile diarrhea, EHEC, an emerging zoonotic pathogen causes diarrhea that can lead to hemorrhagic colitis and hemolytic uremic syndrome. EPEC, ETEC, EIEC and EHEC, as well as other known bacterial pathogens, such as Salmonella and Shigella, for example, remain serious threats to human health.

As such, there still exists a need for improving the prevention and/or treatment of enteropathogenic infection of the gastrointestinal tract and diarrhea associated with infection.

### BRIEF SUMMARY OF THE INVENTION

In accordance with an embodiment, the present invention provides a method for prophylaxis and treatment of diarrhea due to enteropathogenic bacterial infection in the gastrointestinal tract of a subject, the method comprising administering to the subject an effective amount of a low molecular weight polyethylene glycol (PEG).

In an embodiment, the low molecular weight PEG suitable for use in the above method of treatment, has a molecular weight between about 100 Daltons and 5000 Daltons, preferably between about 500 Daltons and 3,500 Daltons, and most preferably about 3350 Daltons.

In accordance with another embodiment, the present invention provides that an effective amount of low molecular weight PEG administered to the subject is between about 1 gram to about 25 grams/day of a low molecular weight PEG, preferably between about 10 grams to about 20 grams/day of a low molecular weight PEG, and most preferably about 17 grams/day of a low molecular weight PEG.

It is also contemplated, that in an embodiment of the present invention, the low molecular weight PEG is administered to the subject prior to infection with enteropathogenic bacteria, for example, between about 1 day to about 14 days prior to infection, preferably at least about 7 days prior to infection.

It is further contemplated, that in an embodiment of the present invention, administration of the low molecular weight PEG to the subject commences or continues to be administered to the subject, for between about 1 day to about 14 days post-infection with enteropathogenic bacteria, preferably at least for about 7 days post-infection.

In accordance with another embodiment, the present invention provides a method for reducing or suppressing inflammation in the gastrointestinal tract of a subject, the method comprising administering to the subject an effective amount of a low molecular weight PEG.

In accordance with a further embodiment, the present invention provides a method for reducing or inhibiting Epidermal Growth Factor Receptor (EGFR) expression and/or phosphorylation in the gastrointestinal tract of a subject, the method comprising administering to the subject an effective amount of a low molecular weight PEG.

In accordance with a further embodiment, the present invention provides a method for reducing or inhibiting β1-integrin expression in the gastrointestinal tract of a subject, the method comprising administering to the subject an effective amount of a low molecular weight PEG.

In another embodiment, the present invention comprises a method for prophylaxis and treatment of diarrhea due to enteropathogenic bacterial infection in the gastrointestinal tract of a subject, further comprising administering to a subject one or more additional agents selected from the group consisting of antidiarrheal agents and antibiotics.

### BRIEF DESCRIPTION OF THE SEVERAL VIEWS OF THE DRAWING(S)

FIG. 1 is a graph depicting the results of immunofluorescent staining of bacterial LPS. The staining revealed that C. rodentium attachment to CMT-93 monolayers was decreased with treatment and pretreatment with PEG (magnification ×60). The number of LPS stained bacterial colonies was recorded in 15 different fields per each experimental group (*p<0.05).

FIG. 2A is a photograph of representative mouse colons from the different treatment groups. The appearance of colon was assessed between different experimental groups 14 days post infection. In contrast to C. rodentium infection, treatment or pretreatment with PEG resulted in stool formation that was similar to control.

FIG. 2B is a graph showing change in colon weight. The length and weights of colons cleaned of stool and extensively washed were expressed as g/cm and expressed as % of change relative to control. Increased colonic weight due to C. rodentuum infection was reduced by PEG.

FIG. 2C is a graph depicting the number of colony forming units (CFU) per gram of mucosa protein. Colons of different experimental groups were cleaned and scraped mucosa was dissolved in lysis buffer. The aliquots were plated on MacConkey plates and the number of colonies was significantly lower with PEG or pretreatment with PEG (, n=6-9, *p<0.05).

FIG. 3 is a series of histological sections and a graph showing that PEG inhibits inflammation in the gut after infection by C. rodentium. Colonic inflammation was assessed in H&E stained tissue and scored by a pathologist. Treatment with PEG significantly decreased colonic inflammation induced by C. rodentium infection, while pretreatment with PEG was sufficient to lower colonic inflammation (n=6-9, *p<0.05).

FIG. 4 is an immunoblot and graph showing that the signaling pathways induced by C. rodentium infection were inhibited by PEG. Protein from scraped colonic mucosa of different experimental groups was immunoblotted for EGFR and phosphorylated EGFR. Increased EGFR expression and phosphorylation in C. rodentium infected colon was inhibited by the presence of PEG. The graph shows densitometric analysis (n=4, *p<0.05).

FIG. 5A is graph depicting the number of colonies of C. rodentium per gram of protein, 7 days post-infection.

FIG. 5B is a graph depicting the number of colonies per gram of protein 14 days post-infection. Both FIGS. 5A and 5B show a that shedding of C. rodentium into the stool is significantly lessened after treatment with PEG.

FIG. 6A is an immunoblot of protein from intestinal HT-29 cells treated with PEG for various times (0, 1, 2, 4, 6, and 24 hours) using antibodies against β1-integrin and actin (loading control). During PEG treatment, the amount of β1-integrin decreased in HT-29 cells.

FIG. 6B is a graph which shows that 5% and 10% PEG significantly attenuated bacterial attachment to CMT-93 monolayers. CMT-93 monolayers were incubated with different concentrations of PEG for 3 hours and infected with C. rodentium for 5 hours in the presence of PEG. Diluted aliquots of cell lysates were plated on LB agar plates. This experiment was repeated three times, and one representative experiment is shown (n=4, *p<0.05).

FIG. 6C is a graph depicting the bacterial growth of C. rodentium in the presence or absence of PEG. C. rodentium culture diluted in fresh tissue medium (serum and antibiotic free) was grown in presence of 1, 5, and 10% PEG. Bacterial growth, as determined by OD 660, was not affected by the presence of PEG (n=6).

### DETAILED DESCRIPTION OF THE INVENTION

It has now been found that enteropathogenic bacterial infections of the gastrointestinal mucosa of a subject can be reduced, suppressed or inhibited by administration of an effective amount of a low molecular weight PEG to the subject prior to, or concurrently with, an infection by enteropathogenic bacteria,

In accordance with an embodiment, the present invention provides a method for prophylaxis and treatment of diarrhea due to enteropathogenic bacterial infection in the gastrointestinal tract of a subject, the method comprising administering to the subject an effective amount of a low molecular weight PEG.

In an embodiment, the present invention provides that the low molecular weight PEG used in the above method of treatment, has a molecular weight between about 100 Daltons and 5000 Daltons, preferably between about 500 Daltons and 3,500 Daltons, and most preferably about 3350 Daltons.

In accordance with another embodiment, the present invention provides that the effective amount of low molecular weight PEG administered to the subject is between about 1 gram to about 25 grams/day of a low molecular weight PEG, preferably between about 10 grams to about 20 grams/day of a low molecular weight PEG, and most preferably about 17 grams/day of a low molecular weight PEG.

It is also contemplated, that in an embodiment of the present invention, the low molecular weight PEG is administered to the subject prior to infection with enteropathogenic bacteria, for example, between about 1 day to about 14 days prior to infection, preferably at least about 7 days prior to infection.

It is further contemplated, that in an embodiment of the present invention, administration of the low molecular weight PEG to the subject commences or continues to be administered to the subject, for between about 1 day to about 14 days post-infection with enteropathogenic bacteria, preferably at least for about 7 days post-infection.

In accordance with another embodiment, the present invention provides a method for reducing or suppressing inflammation in the gastrointestinal tract of a subject, the method comprising administering to the subject an effective amount of a low molecular weight PEG.

In accordance with a further embodiment, the present invention provides a method for reducing or inhibiting Epidermal Growth Factor Receptor (EGFR) expression and/or phosphorylation in the gastrointestinal tract of a subject, the method comprising administering to the subject an effective amount of a low molecular weight PEG.

In accordance with a further embodiment, the present invention provides a method for reducing or inhibiting β1-integrin expression in the gastrointestinal tract of a subject, the method comprising administering to the subject an effective amount of a low molecular weight PEG.

In another embodiment, the present invention comprises a method for prophylaxis and treatment of diarrhea due to enteropathogenic bacterial infection in the gastrointestinal tract of a subject, further comprising administering to a subject one or more additional agents selected from the group consisting of antidiarrheal agents and antibiotics.

In an embodiment, the present invention provides a method for treatment wherein the route of administration is oral or rectal or through oral gavage. The route of administration of PEG, in accordance with the present invention, is in accord with known methods, e.g., oral ingestion, etc.

The term “treat” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the inventive methods can provide any amount of any level of treatment of enteropathic bacterial infection in a subject. Furthermore, the treatment provided by the inventive method can include treatment of one or more conditions or symptoms of the enteropathic bacterial infection being treated, including, for example, diarrhea, hemorrhagic colitis, and hemolytic uremic syndrome.

An effective amount of PEG to be employed therapeutically will depend, for example, upon the therapeutic and treatment objectives, the route of administration, the age, condition, and body mass of the patient undergoing treatment or therapy, and auxiliary or adjuvant therapies being provided to the patient. Accordingly, it will be necessary and routine for the practitioner to titer the dosage and modify the route of administration, as required, to obtain the optimal therapeutic effect. A typical daily dosage might range from at least about 1 g/day up to about 10 g/day for children, for example, from 1 gram/day to up to about 25 g/day of PEG or more for adults. In adults, the dosage is preferably from about 10 g/day to about 20 g/day of PEG depending on the above-mentioned factors. Typically, the clinician will administer PEG until a dosage is reached that achieves the desired effect. The progress of this therapy is easily monitored by observation of the subject being treated.

The dosage ranges for the administration PEG are those large enough to produce the desired effect in which the gastrointestinal symptoms, for example, diarrhea, bloody stool, cramping or bloating are ameliorated. The dosage should not be so large as to cause adverse side effects, such as unwanted cross-reactions, anaphylactic reactions, and the like. Generally, the dosage will vary with the age, condition, sex, and extent of disease of the patient and can be determined by one of skill in the art. The dosages can be adjusted by the individual physician, in the event of any complication. In an embodiment, the methods of the present invention provide for the administration of PEG to children to prevent or reduce infection from enteropathogenic bacteria, ranging in age from six months to 16 years old. In another embodiment, the methods of the present invention provide for the administration of PEG to adults to prevent or reduce infection from enteropathogenic bacteria.

PEG can be administered orally, rectally, or by gavage, alone or in combination with other drugs. Aqueous carriers and vehicles include, for example, water, alcoholic/aqueous solutions, emulsions or suspensions, including saline and buffered media. Preservatives and other additives may also be present such as, for example, antimicrobials, anti-oxidants, chelating agents, and inert gases and the like.

Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

In an embodiment, PEG is given orally in at least one dose, as an aqueous suspension that is consumed in at least one sitting.

Formulations suitable for oral administration can consist of (a) liquid solutions, such as an effective amount of the PEG dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant.

Lozenge and/or mouth wash forms can comprise PEG in a flavor, usually sucrose and acacia or tragacanth, as well as pastilles comprising the inventive TCR material in an inert base, such as gelatin and glycerin, or sucrose and acacia, emulsions, gels, and the like containing, in addition to, such excipients as are known in the art.

PEG, alone or in combination with other suitable components, can be made into aerosol formulations, and also may be formulated as pharmaceuticals for non-pressured preparations, such as in a nebulizer or an atomizer Such spray formulations also may be used to spray mucosa.

PEG can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Preservatives and buffers may be used.

The term “polyethylene glycol” refers to poly(ethylene glycol) (PEG), otherwise known as poly(oxyethylene) or poly(ethylene oxide) (PEO), and is a synthetic polyether that is readily available in a range of molecular weights. Materials with molecular weights under 100,000 Daltons are usually called PEGs, while higher molecular weight polymers are classified as PEOs. These polymers are amphiphilic and soluble in water as well as in many organic solvents (e.g., methylene chloride, ethanol, toluene, acetone, and chloroform). Lower molecular weight (<5,000 Daltons) PEGs are viscous and colorless liquids, while higher molecular weight PEGs are waxy, white solids with melting points proportional to their molecular weights to an upper limit of about 67° C. Low molecular weight PEGs are available at weights of 190 Daltons, 300 Daltons, 400 Daltons and higher (up to about 5000 Daltons). For purposes of the present invention, the term “PEG,” as used herein is synonymous with a low molecular weight PEG.

PEG is the active ingredient of a number of commercially available laxatives (e.g., macrogol-containing products such as Movicol® and polyethylene glycol 3350, or SoftLax®, MiraLAX® or GlycoLax®). Whole bowel irrigation (polyethylene glycol with added electrolytes) is used for bowel preparation before surgery or colonoscopy. It is sold under the brand names GoLYTELY®, GaviLyte-C®, NuLytely®, GlycoLax®, Fortrans®, TriLyte®, Colyte®, Halflytely®, Softlax®, ClearLax® and MoviPrep®. In the United States, MiraLAX®, amd Dulcolax Balance® are sold without prescription for short term relief of chronic constipation.

The terms “prophylaxis” or “prevention” as used herein mean that application of the methods of the embodiments of the present invention can be performed prior to a subject being exposed or infected by enteropathogenic bacteria. For use to prophylactically prevent enteropathogenic bacteria from infecting the gastrointestinal tract of a subject, PEG is administered to the subject prior to infection. Preferably, in accordance with an embodiment of the present invention, PEG is administered to the subject at least 1 to 14 days prior to infection. More preferably, PEG is administered to the subject at least 3 to 10 days prior to infection. Even more preferably, PEG is administered to the subject at least 5 to 8 days prior to infection. In an embodiment, PEG is administered to the subject at least 7 days prior to infection. However, given the non-toxic nature of PEG, the number of days of treatment of a subject prior to infection need not be limited to the time periods listed herein.

For purposes of the present invention, the term “treatment” means administering to a subject already infected with an enteropathogenic bacteria, an effective amount of PEG. Preferably, in accordance with an embodiment of the present invention, PEG is administered to the subject at least 1 to 14 days after infection. More preferably, PEG is administered to the subject at least 3 to 10 days after infection. Even more preferably, PEG is administered to the subject at least 5 to 8 days after infection. In an embodiment, PEG is administered to the subject at least 7 days after infection. However, given the non-toxic nature of PEG, the number of days of treatment of a subject after infection should not be limited to the extent that no adverse side-effects are observed.

The present invention also contemplates, in an embodiment, pharmaceutical compositions comprising low molecular weight PEG and one or more other agents for use in the methods of the present invention. In an embodiment, the present invention provides a pharmaceutical composition comprising PEG and one or more antibiotic agents, and/or one or more anti-diarrheal agents, and a pharmaceutically acceptable carrier.

Examples of antibiotic agents suitable for use in pharmaceutical composition comprising PEG and one or more antibiotic agents include, for example, quinolone antibiotics, such as levofloxacin, ciprofloxacin, ibafloxacin, pradofloxacin, rosoxacin, and sarafloxacin. Other suitable antibiotics are trimethoprim-sulfamethoxazole mixtures such as Bactrim®. Alternatives include rifaximin and azithromycin. Dosages vary with the weight and age of the subject to be treated. Typically, quinolone antibiotics and trimethoprim-sulfamethoxazole mixtures are given at dosages between 250 and 500 mg daily. For trimethoprim-sulfamethoxazole, the dosages are generally between about 5 mg/kg and 25 mg/kg. For rifaximin the dosage ranges from 100 mg to about 500 mg, with 200 mg being preferred. Azithromycin is typically administered at 250-500 mg/day. The dosages required are well within the knowledge of those of ordinary skill in the art.

Other agents included in the pharmaceutical compositions suitable for use in the methods of the present invention include antidiarrheal agents. Examples of such agents include, but are not limited to, bismuth subsalicylate, loperamide, diphenoxylate, and kaolin suspensions.

For purposes of the present invention, the term “diarrhea,” as used herein means loose, watery stools of a subject. A subject having diarrhea means the subject is passing loose stools at least three or more times a day. The term “acute diarrhea” is a common problem that usually lasts 1 or 2 days. Diarrhea lasting more than 2 days is often a sign of an enteropathogenic infection. The term “chronic diarrhea” means diarrhea that lasts at least 4 weeks, and may be a symptom of a chronic disease. Chronic diarrhea symptoms may be continual or intermittent. The term “traveler's diarrhea” means diarrheal symptoms associated with travel-related infection. It may be caused by many different organisms, including bacteria such as E. coli, Salmonella, Shigella, Campylobacter, Aeromonas, Plesiomonas, and vibrios; parasites such as Giardia, Entamoeba histolytica, Cryptosporidium, and Cyclospora; and viruses. In addition to diarrhea, symptoms may include nausea, vomiting, abdominal pain, fever, sweats, chills, headache, and malaise. Diarrhea may also be the result of food borne enteropathogens. Typical food borne pathogens are E. coli, Salmonella, Shigella, Yersinia, and Campylobacter.

Diarrhea of any duration may cause dehydration, which means the body lacks enough fluid and electrolytes—chemicals in salts, including sodium, potassium, and chloride—to function properly. Loose stools contain more fluid and electrolytes and weigh more than solid stools.

In accordance with an embodiment, the present invention provides a method for reducing or suppressing inflammation in the gastrointestinal tract of a subject by administering to the subject an effective amount of a low molecular weight polyethylene glycol. It is contemplated that PEG can be used to treat or suppress symptoms of inflammation in the gastrointestinal tract of a subject. Examples of symptoms of such inflammation include, but are not limited to, architectural distortion of the gastrointestinal mucosal tissues and increased lamina propria lymphocytes, increased lamina propria granulocytes in the gastrointestinal mucosa, the presence of intraepithelial granulocytes in the gastrointestinal mucosa with, or without, crypt abscesses, and erosion/ulceration of the gastrointestinal mucosa. It is therefore contemplated that the methods of the present invention are useful for prevention or treatment of inflammatory bowel disease, Crone's disease, ulcerative colitis and other immune system related diseases of the gastrointestinal tract.

For treatment of inflammation in the gastrointestinal tract of a subject, the daily dosage ranges from at least about 1 g/day to up to about 25 g/day of PEG or more, preferably from about 10 g/day to about 20 g/day of PEG. Preferably, in accordance with an embodiment of the present invention, PEG is administered to the subject at least 1 to 14 days, however PEG can be administered for a longer period, or until the inflammatory symptoms are alleviated.

In accordance with another embodiment, the present invention provides a method for reducing or inhibiting EGFR expression and/or phosphorylation in the gastrointestinal tract of a subject, by administering to the subject an effective amount of a low molecular weight polyethylene glycol. The dosages and duration of use is dependent on the amount of reduction of EGFR expression or phosphorylation desired. The daily dosage might range from at least about 1 g/day to up to about 25 g/day of PEG or more, preferably from about 10 g/day to about 20 g/day of PEG. Preferably, in accordance with an embodiment of the present invention, PEG is administered to the subject at least 1 to 14 days, however PEG can be administered for a longer period, or until the reduction of expression and/or phosphorylation of EGFR in the gastrointestinal mucosa is achieved.

In accordance with a further embodiment, the present invention provides a method for reducing or inhibiting β1-integrin expression in the gastrointestinal tract of a subject by administering to the subject an effective amount of a low molecular weight polyethylene glycol. The dosages and duration of use is dependent on the amount of reduction of β1-integrin expression desired. The daily dosage might range from at least about 1 g/day to up to about 25 g/day of PEG or more, preferably from about 10 g/day to about 20 g/day of PEG. Preferably, in accordance with an embodiment of the present invention, PEG is administered to the subject at least 1 to 14 days, however PEG can be administered for a longer period, or until the reduction of expression of β1-integrin in the gastrointestinal mucosa is achieved.

In accordance with a further embodiment, the present invention provides a kit suitable for use in the methods of the present invention. The kit, for example, comprises at least one dose of a low molecular weight PEG in, for example, dry form, and instructions for preparing the at least one dose of PEG for oral administration and its use in the prevention and/or treatment of enteropathogenic bacterial infection. In another embodiment, the kit comprises between about 7, to about 14 doses of a low molecular weight PEG in dry form, and instructions for preparing the doses of PEG for oral administration, and their use in the prevention and/or treatment of enteropathogenic bacterial infection.

### EXAMPLES

The following examples further illustrate the invention but, of course, should not be construed as in any way limiting its scope.

Tissue culture. Mouse colonic CMT-93 cells (American Type Culture Collection (ATCC)), were propagated in DMEM medium (Gibco, Carlsbad, Calif.) with 10% FBS (Gibco) at 37° C. with 5% CO₂ incubator. Human colon HT-29 cells (ATCC) were propagated in McCoy's 5A medium (Sigma, St. Louis, Mo.) supplemented with 10% FBS (Gibco). Cell monolayers were serum deprived overnight prior to experiments.

PEG treatment. For in vitro studies, 5% PEG (MW=3,350) (Sigma, St. Louis, Mo.) dissolved in serum free antibiotic free DMEM was added to monolayers three hours before infection. For in vivo studies, 5% PEG (200 μl) was introduced daily by oral gavages to mice for seven days prior to infection. One experimental group continually received PEG daily during infection.

C. rodenitum infection in vitro. C. rodentium cultures were grown overnight in Luria Bertani broth (LB) at 37° C. Bacteria were diluted (1:33) in DMEM (serum free, antibiotics free) and allowed to grow to mid-log growth phase (OD 660 nm˜0.5) overnight. Bacterial pellets, were resuspended in serum and antibiotic-free DMEM, was applied to 100 monolayers (100 bacteria/cell) and incubated at 37° C. in 5% CO₂.

C. rodentium infection in vivo. For in vivo studies C57BL/6J mice (4-6 weeks old) were used. Mice were infected with C. rodentium as previously described (Snoeks, L., et al., Infect. Immun., 76:4677-4685 (2008)) in accordance with approved animal care protocols of Northshore University Healthsystem Research Institute.

Bacterial colony counts. Monolayers of cells infected with bacteria were washed five times with PBS, lysed with 1% Triton X-100, and diluted aliquots plated on LD agar plates. For in vivo experiments, scraped colonic mucosa was lysed and the fresh stool particles were homogenized in PBS, and aliquots were plated on MacConkey agar plates. C. rodentium colonies were recognized as pink with a white rim after incubation overnight at 37° C. C. rodentium colonies grown on LB or MacConkey agar plates were randomly selected and confirmed by PCR as previously described (Savkovic, S. D., et al., Infect. Immun., 73:1161-1170 (2005)).

Immunofluorescent staining. Infected monolayers were extensively washed and fixed with 3.7% paraformaldehyde. Monolayers were incubated with anti-LPS antibody (Santa Cruz Biotechnology, Calif.) following incubation with Alexa 488 conjugated secondary antibody (Molecular Probes-Invitrogen, Carlsbad, Calif.). Coverslips were mounted using Prolong Gold antifade reagent (Molecular Probes) and images were captured using a Nikon Confocal Microscope C1, and analyzed with EZ-C1 software (Nikon, Tokyo, Japan).

Protein extraction. Total protein was extracted from scraped mucosa using cell lysis buffer (Cell Signaling) supplemented with a protease inhibitor cocktail (Sigma). Protein concentration was determined using the Bradford assay (Bio-Rad, Hercules, Calif.) and aliquots were stored at −20° C.

Immunoblots. Total protein (40 μg) was separated by SDS-PAGE and transferred to nitrocellulose membranes (Bio-Rad), as previously described (Qi, W., et al., Am. J. Physiol. Gastrointest. Liver Physiol., 300:G264-272 (2011)). Primary antibodies against EGFR, pEGFR, β-actin (Santa Cruz Biotechnology), and β₁-integrin (Cell Signaling) were used. Secondary antibodies linked to horseradish peroxidase (Cell Signaling) were visualized using ECL plus western blotting detection reagents (GE Healthcare, Buckinghamshire, United Kingdom).

Histological analysis. Distal segments of colons were formalin-fixed and paraffin embedded, while the tissue sections (5 μm thick) were stained with hematoxylin and eosin (H&E) staining. The degree of inflammation was evaluated according to the following criteria: 0—no architectural distortion or infiltrates; 1—architectural distortion, increased lamina propria lymphocytes, no activity; 2—increased lamina propria granulocytes without definite intraepithelial granulocytes (i.e. no activity); 3—intraepithelial granulocytes (i.e. activity) without crypt abscesses; 4—crypt abscesses in less than 50% of crypts; 5—crypt abscesses in greater than 50% of crypts or erosion/ulceration.

Statistical analysis. Data were analyzed by Student's t-test and expressed as means±SEM. Differences were considered significant at p<0.05.

### Example 1

This example demonstrates that PEG inhibits the attachment of C. rodentium to mouse colonic cells in vitro.

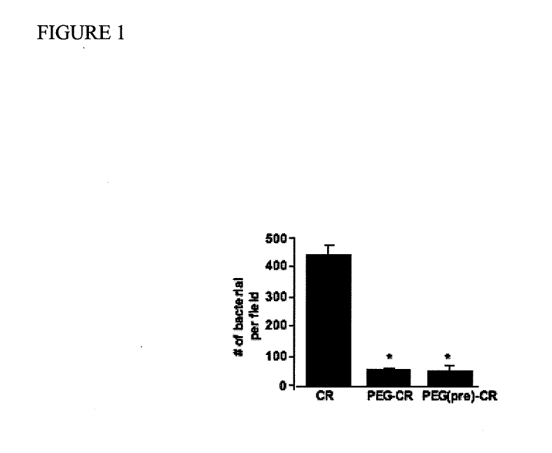

Mouse colonic CMT-93 cells were incubated with a control solution or a solution of 5% PEG, and then they were infected with C. rodentium either with continuous exposure to PEG (PEG+CR) or after removing PEG and washing (PEG (pre)+CR). After 5 hours of exposure, the groups (Control, PEG(only), CR, PEG+CR, and PEG (pre)+CR) were washed extensively and stained with an immunofluorescent dye specific for bacterial lipopolysaccharide (LPS). Photomicrographs were taken after staining (data not shown). The staining showed that the CR group had the greatest number of bacterial colonies, with the (PEG (pre)+CR) group significantly lower. The PEG+CR group had even less colonies. The number of LPS stained bacterial colonies was recorded in 15 different fields per group (FIG. 1). The presence or pretreatment of the colonic cells with PEG significantly reduced bacterial attachment.

### Example 2

This example demonstrates that PEG inhibits the attachment of C. rodentium to mouse colonic cells in vitro.

The effects of PEG on the attachment of C. rodentium to the mouse colon was investigated. Five experimental groups were established. A control group with no treatment. A group infected for 2 weeks with C. rodentium (CR) via daily oral gavage. A group treated only with PEG for 1 week (PEG) via daily oral gavage. Another group was infected with C. rodentium for 2 weeks while also receiving concomitant PEG treatment (PEG−CR). And a final group was treated with PEG via daily oral gavage for 1 week, and then infected with C. rodentium for 2 weeks, without further PEG administration (PEG(pre)−CR). The mice were then euthanized the colons were removed, cleaned of stool and washed. Colonic mucosa was scraped, lysed with 1% Triton X® and diluted aliquots were plated on MacConkey agar plates and incubated with 5% CO₂ at 37° C. overnight. The numbers of colonies which developed on the agar plates were counted and expressed as per gram of mucosal protein. In both groups, PEG(pre)−CR and PEG−CR, C. rodentium attachment to colonic mucosa was significantly attenuated relative to the CR group

### Example 3

This example demonstrates that administration of PEG improved clinical signs of C. rodentium infection.

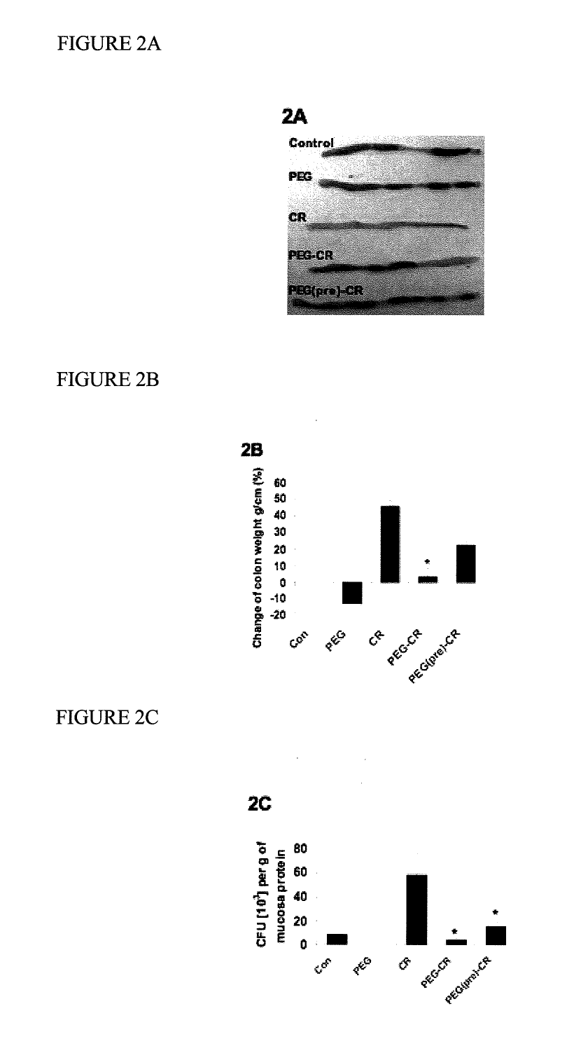

It is known that C. rodentium infection in mice causes diarrhea that is characterized by the absence of solid stool pellets in the colon. The experiment was designed to determine whether treatment or pretreatment of mice with PEG improved stool formation in the colons of mice infected with C. rodentium. The same five experimental groups of mice were established as in Example 2 above. Control mice and mice treated with PEG only were able to form stool pellets in their colons (FIG. 2A). Mice infected with C. rodentium were unable to form stool pellets. Mice treated or pretreated with PEG and then infected with C. rodentium were also able to form stool pellets. Interestingly, the PEG alone group did not induce diarrhea in this study. In addition, colonic weight was increased by 46±3% in the group infected with C. rodentium alone, but was significantly reduced in the groups of mice which received PEG and was partially reduced in the group which was pretreated with PEG. Colonic weight was measured as gram per centimeter of length (FIG. 2B). Furthermore, the number of C. rodentium colonies attached to colonic mucosa of mice receiving PEG is significantly decreased (control=8±1, C. rodentium=58±18, PEG treated and C. rodentium=4±1, PEG pretreated and C. rodentium=15±3 colonies×103/g mucosal protein) (FIG. 2C). Altogether, these data support that PEG can efficiently lower attachment of C. rodentium to the colonic mucosa and improves macroscopic changes of infected colons.

### Example 4

This example illustrates how PEG inhibits C. rodentium induced inflammatory response in colonic mucosa.

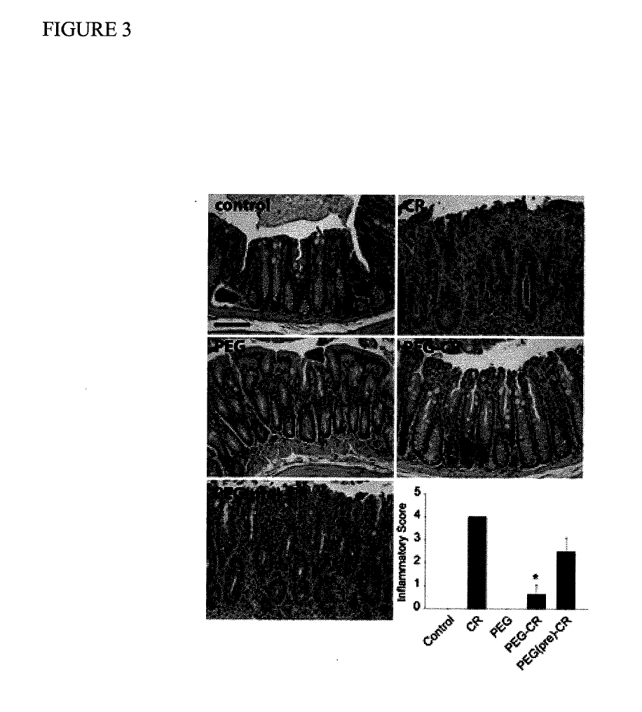

It is known that infection of the colonic mucosa with C. rodentium induces inflammation within the mucosa, and represent the clinical characteristic of infection with E. coli enteric pathogens. A study was therefore undertaken to determine whether C. rodentium induced inflammation in colonic mucosa was attenuated by PEG treatment. After treatment, colonic tissue sections of mice were collected and stained with H&E stain. The sections were then observed by microscopy and the sections were scored using the following criteria: 0—completely uninvolved, no architectural distortion or infiltrates; 1—architectural distortion, increased lamina propria lymphocytes, no activity; 2—increased lamina propria granulocytes without definite intraepithelial granulocytes (i.e., no activity); 3—intraepithelial granulocytes (i.e., activity) without crypt abscesses; 4—crypt abscesses in less than 50% of crypts; 5—crypt abscesses in greater than 50% of crypts or erosion/ulceration. Active colonic inflammation was subjected to blinded scoring by a pathologist. C. rodentium infection induced infiltration of inflammatory cells into the lamina propria and epithelia, and induces formation of crypt abscesses in the mouse colon (activity score=4.0±0) (FIG. 3). In colons of infected mice, concurrently treated with PEG, although there were still lymphocytes present in the lamina propria, active intraepithelial inflammation was not observed (activity score=0.6±0.4). Pretreatment with PEG further decreased inflammation scores, and scores were not significantly elevated compared to uninfected controls (activity score=2.5±0.5). The results show histologically, that treatment with PEG normalizes the C. rodentium induced inflammatory response.

### Example 5

This example shows how PEG inhibits intracellular signaling pathways induced by C. rodentium infection in colonic epithelia.

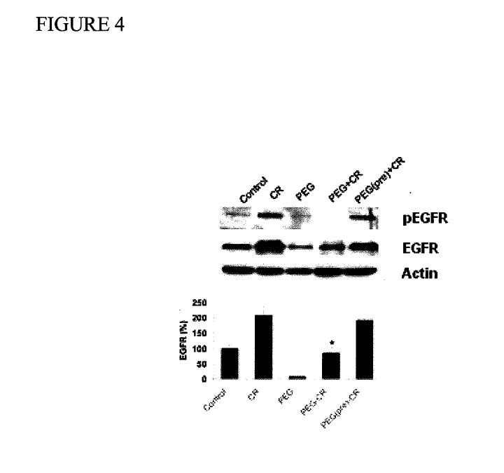

The investigators previously demonstrated that C. rodentium infection increases expression and phosphorylation of Epithelial Growth Factor Receptor (EGFR) in colonic epithelia (Qi, W., et al., supra). A study was undertaken to determine whether PEG inhibited the C. rodentium induced EGFR activation in colonic epithelia. The same five experimental groups of mice were established as in Example 2 above. Protein from scraped colonic mucosa of the different experimental groups were separated and immunoblotted for EGFR and phosphorylated EGFR. Infection with C. rodentium increased EGFR expression 208±23% when compared to controls. Moreover, C. rodentium induced phosphorylation of EGFR was reduced 81±14% by the presence of PEG, and the EGFR expression level was similar to control (FIG. 4). PEG treatment alone, significantly reduced EGFR expression, and pretreatment with PEG modestly lowered C. rodentium increased EGFR expression. These data support the concept that PEG is effective at inhibiting signals generated from the cell membrane induced by C. rodentium infection.

### Example 6

This example discloses that treatment with PEG lowers the amount of C. rodentium shed in the stools of infected mice.

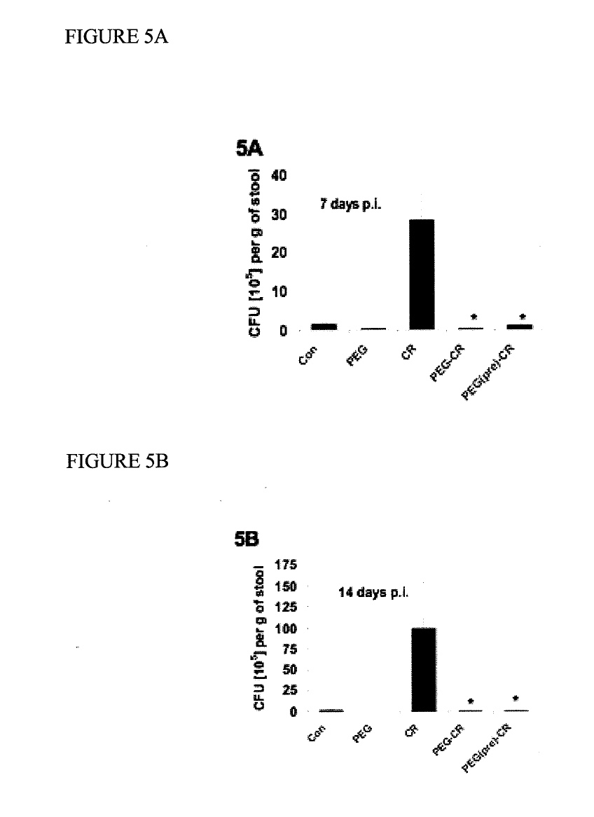

It is known that one of the hallmarks of infection by enteropathogenic bacteria is that they multiply in the infected intestines and spread through the excreted stool and infect other hosts. A study was undertaken to determine whether treatment with PEG would inhibit bacterial shedding of C. rodentium into the stool of infected mice. The same five experimental groups of mice were established as in Example 2 above. Stools were collected from the experimental mice at day 7 post-infection, and dissolved. Diluted aliquots were plated on MacConkey agar plates and incubated overnight. The number of colonies on the plates of each group were counted and expressed as colonies per gram of stool (FIGS. 5A and 5B). The presence of PEG drastically reduced the number of bacterial colonies shed via the stool during C. rodentium infection. Also, pretreatment with PEG showed a decrease in the number of C. rodentium colonies in the stool (7 days: control 1.5±0.3, C. rodentium 28.5±7.8, PEG−C. rodentium 0.6±0.1, PEG(pre)−C. rodentium 1.2±0.4×CFU per g of stool). Thus, the presence or pretreatment with PEG lowers shedding of C. rodentium in the stool supporting that PEG may have a preventive effect on bacterial dispersion, and may help to curtail spread of infection.

### Example 7

This example discloses that treatment with PEG down regulates β1-integrin expression in colonic HT-29 cells in vitro.

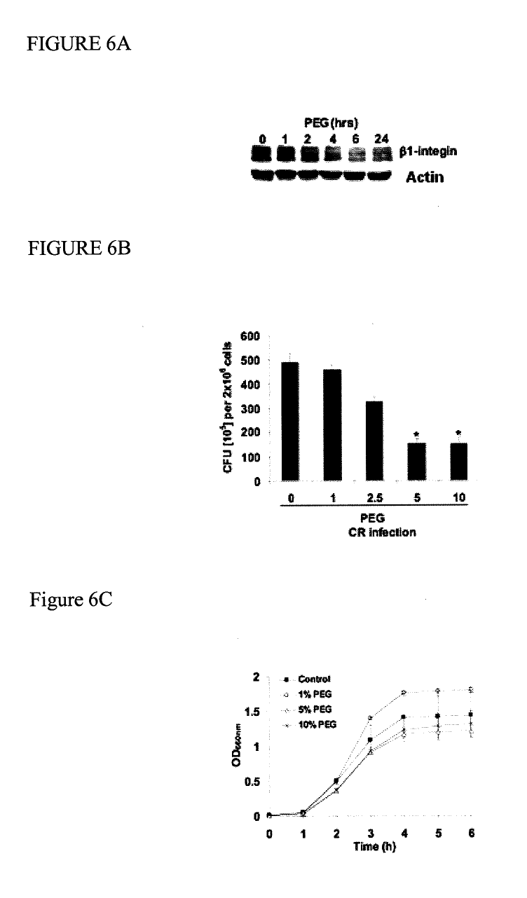

It is known that enteropathogenic bacteria interact with host cellular adhesion molecules such as integrins. Moreover, for E. coli strains EPEC, EHEC and C. rodentium, attachment to intestinal epithelial cells and interaction with β1-integrin are important steps in their pathogenicity. An experiment was developed to determine whether PEG may be involved in down regulation of the expression of β1-integrin, which could, consequently, inhibit the attachment of C. rodentium to the host cells. HT-29 cells were divided into control and PEG treated groups. The cells were exposed to vehicle or PEG (5%) for time points of 0, 1, 2, 4, 6, and 24 hours. Cell monolayers were washed, and the cells scraped and the protein extracted. HT-29 cells treated with PEG for various time points were immunoblotted with antibodies against β1-integrin and actin. The results of this study shows that PEG lowers β1-integin in intestinal cells (FIG. 6A). The presence of PEG decreased C. rodentium attachment to mouse CMT-93 monolayers (control 492±39 and 5% PEG 151±27×105 CFU per 2×106 cells) (FIG. 6B), without affecting bacterial growth (FIG. 6C) or the ability of bacteria to attach (data not shown). Additionally, immunofluorescent staining revealed that pretreatment of CMT-93 monolayers with PEG significantly attenuated C. rodentium attachment (data not shown), suggesting that PEG-induced changes in host cell membrane led to cellular bacterial resistance. These data collectively show that PEG protects mouse intestinal CMT-93 monolayers from C. rodentium attachment by decreasing sensitivity of host cell to the bacteria.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for prophylaxis and treatment of diarrhea due to enteropathogenic bacterial infection in the gastrointestinal tract of a subject, the method comprising:
administering to the subject an effective amount of a low molecular weight polyethylene glycol.

2. The method of claim 1, wherein the low molecular weight polyethylene glycol has a molecular weight between about 100 daltons and 5000 daltons, preferably between about 500 daltons and 5000 daltons.

3. The method of claim 1, wherein the low molecular weight polyethylene glycol has a molecular weight of about 3350 to about 5000 daltons.

4. The method of claim 1, wherein the effective amount administered to the subject is between about 1 grams to about 25 grams/day of a low molecular weight polyethylene glycol, preferably between about 10 grams to about 20 grams/day of a low molecular weight polyethylene glycol.

5. The method of claim 1, wherein the low molecular weight polyethylene glycol is administered to the subject prior to infection with enteropathogenic bacteria.

6. The method of claim 5, wherein the low molecular weight polyethylene glycol is administered to the subject between about 1 day to about 14 days prior to infection with enteropathogenic bacteria.

7. The method of claim 6, wherein the low molecular weight polyethylene glycol is administered to the subject about 7 days prior to infection with enteropathogenic bacteria.

8. The method of claim 1, wherein the low molecular weight polyethylene glycol is administered to the subject prior to infection with enteropathogenic bacteria, and is continued during subsequent infection with enteropathogenic bacteria.

9. The method of claim 8, wherein administration of the low molecular weight PEG to the subject commences or continues to be administered to the subject, for between about 1 day to about 14 days post-infection with enteropathogenic bacteria, preferably at least for about 7 days post-infection.

10. A method for reducing or suppressing inflammation in the gastrointestinal tract of a subject, the method comprising:
administering to the subject an effective amount of a low molecular weight polyethylene glycol.

11. The method of claim 10, wherein the low molecular weight polyethylene glycol has a molecular weight between about 100 daltons and 5000 daltons, preferably between about 500 daltons and 5000 daltons.

12. The method of claim 11, wherein the low molecular weight polyethylene glycol has a molecular weight of about 3350 daltons.

13. The method of claim 11, wherein the effective amount administered to the subject is between about 1 grams to about 25 grams/day of a low molecular weight polyethylene glycol, preferably between about 10 grams to about 20 grams/day of a low molecular weight polyethylene glycol.

14. A method for reducing or inhibiting β1-integrin expression in the gastrointestinal tract of a subject, the method comprising:
administering to the subject an effective amount of a low molecular weight polyethylene glycol.

15. The method of claim 14, wherein the low molecular weight polyethylene glycol has a molecular weight between about 100 daltons and 5000 daltons, preferably between about 500 daltons and 5000 daltons.

16. The method of claim 15, wherein the low molecular weight polyethylene glycol has a molecular weight of about 3350 daltons.

17. The method of claim 14, wherein the effective amount administered to the subject is between about 1 gram/day to about 25 grams/day of a low molecular weight polyethylene glycol, preferably between about 10 grams/day to about 20 grams/day of a low molecular weight polyethylene glycol.

18. The method of claim 1, further comprising administering to a subject one or more additional agents selected from the group consisting of: antidiarrheal agents and antibiotics.

19. The method of claim 1, wherein the enteropathogenic bacterial infection is caused by infection with any one of the following bacterial species: Campylobacter spp., Salmonella spp., Shigella spp., vibrio and Escherichia coli spp.

20. A kit for use in the method of claim 1, wherein the kit comprises at least one or more doses of a low molecular weight PEG in dry form, and instructions for preparing the at least one or more doses of PEG for oral administration and its use in the prevention and/or treatment of enteropathogenic bacterial infection.
